(19) United States
(12) Patent Application Publication
(10) Pub. No.: US 2021/0076985 A1
(43) Pub. Date: Mar. 18, 2021

(54) FEATURE-BASED JOINT RANGE OF MOTION CAPTURING SYSTEM AND RELATED METHODS

(71) Applicant: DePuy Synthes Products, Inc., Raynham, MA (US)

(72) Inventor: Filip Leszko, West Chester, PA (US)

(21) Appl. No.: 17/019,156

(22) Filed: Sep. 11, 2020

Related U.S. Application Data
(60) Provisional application No. 62/899,876, filed on Sep. 13, 2019.

(51) Int. Cl. A61B 5/11; A61B 5/00; G06T 7/00; G06T 7/254

(52) U.S. Cl. CPC A61B 5/1128; A61B 5/4528; G06T 2207/20224; G06T 7/254; G06T 7/0014

(57) ABSTRACT

Feature-based joint range of motion (ROM) capture systems and methods can provide increased quality and quantity of data to assess joint ROM without requiring a patient to seek professional assistance or equipment. ROM systems disclosed herein can include a first pattern coupled to a first portion of patient anatomy on a first side of a joint and a second pattern coupled to a second portion of patient anatomy on a second side of the joint opposite the first side. At least one image containing the first and second patterns and the joint can be captured with an image capture device. The first and second patterns can be detected in the at least one image using a feature-based image recognition algorithm. Based on the detected patterns, at least one ROM metric of the associated joint, e.g., a maximum flexion angle, a maximum extension angle, or a range-of-motion, can be calculated.

### CROSS REFERENCE TO RELATED APPLICATION

This application claims the benefit of U.S. Provisional Application No. 62/899,876, filed Sep. 13, 2019, and entitled “Feature-Based Joint Range of Motion Capturing System,” which is hereby incorporated by reference in its entirety.

### FIELD

The present disclosure relates to devices, systems, and methods for feature-based joint range of motion capture that can, for example, analyze features of one or more images including a joint and determine at least one associated range of motion metric.

### BACKGROUND

Joint range of motion (ROM) can be an important metric used clinically as an indication of post-operative recovery following a surgical procedure, e.g., a joint replacement or arthroscopic repair surgery. Present options for measuring and monitoring post-operative ROM, however, can be restricted in frequency, accuracy, and can involve a time-consuming process for a patient. For example, for patients who undergo a knee or hip replacement or arthroscopic repair surgery (e.g., ligament or meniscus repair), measurements of joint ROM following the surgery can be an important indication of recovery progress or lack thereof. For the knee, ROM is most commonly assessed using a hand-held goniometer. In order to achieve a meaningful measurement, the operator of the goniometer must have strong knowledge of anatomy such that the goniometer can be properly aligned with anatomical landmarks. Accordingly, ROM measurements can be assessed only in the presence of a trained clinician. As such, assessing joint ROM can require a post-operative follow up visit to a doctor or a physical therapy appointment, which can limit and constrain the frequency with which such measurements can be taken. Moreover, accuracy of goniometers can be limited. For example, a short arm goniometer can have a standard deviation of approximately 9 degrees, while a long arm goniometer can have a standard deviation of approximately 5 degrees. Accuracy can also be subject to operator error as a goniometer must be properly placed relative to patient anatomy to achieve a meaningful response.

Another current option for assessing a joint ROM can include sending a patient to a dedicated motion lab for movement analysis to assess the joint ROM. As with goniometer measurements, this process can require making a post-operative appointment and can only be done in the presence of a trained operator or clinician with specialized equipment, such as an array of cameras to view the joint from different angles and specialty software to analyze the captured images. Accordingly, assessing joint ROM at a motion lab can also restrict the frequency with which such measurement can be made, can be time-consuming for the patient, and can be costly due to the significant specialty equipment utilized.

In both cases, measurements of joint ROM can be infrequent and irregular, which can result in sparse data being made available to an orthopedic surgeon or other medical professional for post-operative assessment. Given the limitations on joint ROM measurement, the available joint ROM data may not have sufficient quality or quantity to adequately reveal abnormal patterns in joint recovery, which can limit the clinical relevance of such an assessment as a useful indication of patient recovery. Moreover, known solutions for joint ROM assessment can be burdensome on a patient, requiring travel and time to a specific location for undergoing the ROM measurement.

Accordingly, there is a need for improved systems, methods, and devices that provide an accessible way to measure joint ROM with greater frequency and accuracy for determining and monitoring post-operative patient recovery.

### SUMMARY

Feature-based joint range of motion capture systems and related methods are disclosed herein for increasing frequency, accuracy, and ease of assessing ROM of a joint of a patient, e.g., following a surgical procedure. Embodiments described herein can provide an accessible, low-cost solution for capturing joint ROM that can be used by patients at home on a daily basis, or as frequently as desired, without the need for a trained operator or clinician. For example, systems in accordance with the present disclosure can include a first pattern that can be coupled to a first portion of patient anatomy on a first side of a joint and a second pattern that can be coupled to a second portion of patient anatomy on a second side of the joint opposite the first. An image capture device, e.g., a mobile device such as a smartphone or a tablet, can capture at least one image including the joint, the first pattern, and the second pattern. Importantly, the patient can couple the first and second patterns to the appropriate patient anatomy and capture the at least one image without assistance from a trained professional and from almost any location. The at least one image can be transmitted to a processor, which can detect the first pattern and the second pattern in the image, calculate axes of the first and second portions of anatomy based on the detected patterns, calculate an angle between the axes in the at least one image, and calculate a range of motion metric based on the calculated angle between the axes. Accordingly, the systems and methods provided herein can provide for an increased frequency of accurate measurements of a joint ROM, without requiring patient appointments with, or travel to, a trained operator to administer the measurement.

In one aspect, a system for measuring joint range of motion can include a first pattern, a second pattern, an image sensor, and a processor. The first pattern can be configured to be coupled to a first portion of anatomy of a patient on a first side of a joint and the second pattern can be configured to be coupled to a second portion of patient anatomy on a second side of the joint opposite the first side. The image sensor can be configured to capture at least one image containing the joint, the first pattern, and the second pattern. The processor can be configured to recognize the first pattern and the second pattern in the at least one image, calculate axes of the first and second portion of anatomy to which the first and second patterns are coupled, calculate an angle between the axes, and calculate at least one range of motion metric of the joint based on the calculated angle between the axes in the at least one image.

The devices and methods described herein can have a number of additional features and/or variations, all of which are within the scope of the present disclosure. In some embodiments, for example, the image sensor and the processor can be contained within a smartphone or a tablet. The range of motion metric can be a full range of motion and the at least one image can include a first image where the joint is at a maximum extension and a second image where the joint is at maximum flexion. The processor can be configured to calculate the full range of motion as a difference between the angle between the axes when the joint is at maximum extension and the angle between the axes when the joint is at maximum flexion. In some embodiments, the range of motion metric can be a maximum extension angle and the at least one image can include an image where the joint is at maximum extension. The processor can be configured to calculate the maximum extension angle as the angle between the axes when the joint is at maximum extension. Alternatively, the range of motion metric can be a maximum flexion angle and the at least one image includes an image where the joint is at maximum flexion. The processor can be configured to calculate the maximum flexion angle based on the angle between the axes when the joint is at maximum flexion.

The first pattern can be coupled to a first elastic band configured to be placed over the first portion of anatomy and the second pattern can be coupled to a second elastic band configured to be placed over the second portion of anatomy. In other embodiments, the first patter can be disposed on a proximal portion of an elastic sleeve and the second pattern can be disposed on a distal portion of the elastic sleeve, with the elastic sleeve configured to be placed over the joint, the first portion of anatomy, and the second portion of anatomy. In some instances, the first portion of anatomy can be a shin, the second portion of anatomy can be a thigh, and the joint can be a knee. Optionally, at least one of the first pattern and the second pattern can include at least one patient-specific marker and the processor can be further configured to identify the patient-specific marker with a particular patient. The processor can communicate with an associated database based on the identified patient.

In another aspect, a method for measuring joint range of motion can include coupling a first pattern to a first portion of anatomy of a patient on a first side of a joint, coupling a second pattern to a second portion of anatomy of the patient on a second side of the joint opposite the first side, and capturing at least one image containing the joint, the first pattern, and the second pattern. The method can include detecting the first pattern and the second pattern for the at least one image, calculating axes of the first and second portions of anatomy based on the detected patterns in the at least one image, calculating an angle between the axes in the at least one image, and calculating a range of motion metric based on the calculated angle between the axes in the at least on image.

The step of detecting the first and second pattern for the at least one image can be performed using a feature-based image recognition algorithm. A smartphone or a tablet can be used to capture the at least one image. The smartphone or tablet can be used to detect the first and second patterns in the at least one image, calculate the axes of the first and second portions of anatomy, calculate the angle between the axes, and calculate the range of motion metric. In some embodiments, capturing the at least one image can include capturing a video segment using the smartphone or tablet. In some embodiments, the first portion of anatomy can be a shin, the second portion of anatomy can be a thigh, and the joint can be a knee.

In some embodiments, capturing the at least one image can further include capturing a first image in which the joint is at maximum extension and a second image in which the joint is at maximum flexion. In some such embodiments, the range of motion metric can be a full range-of-motion and calculating the range of motion metric can further include calculating an angle between the axes of the first image, calculating an angle between the axes of the second image, and calculating the range of motion as a difference between the angle between the axes when the joint is at maximum extension and the angle between the axes when the joint is at maximum flexion. In some embodiments, the at least one image can include an image in which the joint is at maximum extension and the range of motion metric can be a maximum extension. Calculating the maximum extension can include calculating the maximum extension as the angle between the axes when the joint is at maximum extension. The at least one image can include an image in which the joint is at maximum flexion and the range of motion metric can be a maximum flexion. Calculating the maximum flexion can include calculating the maximum flexion as the angle between the axes when the joint is at maximum flexion.

In another aspect, a system for capturing a joint range of motion can include one or more processors of a range of motion platform on a network. The one or more processors can be configured to receive at least one image taken with a smartphone or tablet, the image including a joint, a first pattern coupled to a first portion of anatomy on a first side of the joint, and a second pattern coupled to a second portion of anatomy on a second side of the joint opposite the first side. The one or more processor can detect the first pattern and the second pattern for the at least one image, calculate axes of the first and second portions of anatomy based on the detected patterns in the at least one image, calculate an angle between the axes in the at least one image, and calculate a range of motion metric based on the calculated angle between the axes in the at least one image.

In some embodiments, the range of motion metric can be a range of motion and the at least one image can include a first image where the joint is at a maximum extension and a second image where the joint is at maximum flexion. The one or more processors can calculate the full range of motion as a difference between the angle between the axes when the joint is at maximum extension and the angle between the axes when the joint is at maximum flexion. The first pattern and the second pattern can include at least one patient-specific marker. In some embodiments, the one or more processors can identify the patient-specific marker with a particular patient and communicate with an associated database based on the identified particular patient.

Any of the features or variations described above can be applied to any particular aspect or embodiment of the present disclosure in a number of different combinations. The absence of explicit recitation of any particular combination is due solely to the avoidance of repetition in this summary.

### BRIEF DESCRIPTION OF THE DRAWINGS

FIG. 1 is an illustration of an embodiment of a feature-based joint range of motion (ROM) system of the present disclosure including a patient-wearable device, an image capture device, and an ROM analyzer;

FIG. 1A is a block diagram of one embodiment of the ROM analyzer of the ROM system of FIG. 1;

FIG. 2A is an illustration of another configuration of the joint ROM system of FIG. 1;

FIG. 2B is one example of an image captured by an image capture device of the configuration of FIG. 2A;

FIG. 3 is another example of an image captured by an image capture device in accordance with the joint ROM system of FIG. 1;

FIG. 4 is another embodiment of a patient-wearable device that can be used in accordance with the joint ROM system of FIG. 1;

FIG. 5 is another embodiment of a patient-wearable device that can be used in accordance with the joint ROM system of FIG. 1;

FIG. 6 is one example of an image captured by an image capture device of the patient-wearable device of FIG. 4 in accordance with the joint ROM system of FIG. 1;

FIG. 7 illustrates examples of patterns that can be used in accordance with the ROM system of FIG. 1;

FIG. 8A shows examples of feature-based image recognition that can be used in conjunction with the joint ROM system of FIG. 1;

FIG. 8B shows further examples of feature-based image recognition that can be used in conjunction with the joint ROM system of FIG. 1; and

FIG. 9 is a flowchart of one embodiment of a method of determining one or more joint ROM metric in accordance with the present disclosure.

### DETAILED DESCRIPTION

Feature-based joint range of motion (ROM) capture systems and related methods are disclosed herein that can improve the quality and quantity of joint ROM data to allow for more accurate and effective assessment of a post-operative condition of a patient, e.g., by providing systems and methods that a patient can easily use from home with inexpensive and accessible hardware to capture relevant data and assess one or more range of motion metric based on the captured data.

Joint ROM systems of the present disclosure can include a first pattern, a second pattern, an image sensor, and a processor. The first and second patterns can include one or more elements, such as a geometric shapes, images, lettering, etc., such that features in the pattern can be detected in an image to locate the pattern, e.g., with a feature-based image recognition algorithm. The first pattern can be coupled to a first portion of patient anatomy on a first side of a joint and the second pattern can be coupled to a second portion of patient anatomy on a second side of the joint opposite the first side. The first pattern and the second pattern can be provided in a manner that can allow a patient to securely and removably couple the first and second pattern to the first and second portions of anatomy, respectively, i.e., on opposite sides of a joint. The image sensor can capture at least one image containing the joint, the first pattern coupled to the first portion of anatomy, and the second pattern coupled to the second portion of anatomy. The image sensor can be included in a mobile device, such as a smartphone or a tablet, that can capture the at least one image without requiring professional or specialized instruments or assistance. In other words, the patient can self-sufficiently capture the at least one image containing the joint, the first pattern coupled to the first portion of anatomy, and the second pattern coupled to the second portion of anatomy. The image can be transmitted to the processor, which can be locally accessible (e.g., the processor of mobile device that includes the image sensor) or remotely accessible over a network (e.g., a processor included in a server accessible to the mobile device via a network connection). The processor can recognize the first pattern and the second pattern in the frame of the image, e.g., using the feature-based recognition algorithm, calculate axes of the first and second portion of anatomy to which the first and second patterns are coupled in the image, and calculate an angle between the axes. At least one range of motion metric of the joint can be calculated based, at least in part, on the calculated angle between the axes in the at least one image. As used herein, a range of motion metric can include an angle of maximum extension of a joint, an angle of maximum flexion of a joint, a full range of motion of a joint, which can be calculated as a difference between the angle of maximum extension and flexion, each of which can be used by a surgeon or other medical professional to assess a recovery and/or functioning of a joint. For example, in some instances a surgeon may be interested primarily in the maximum flexion angle of a joint, while in other cases the surgeon may wish to determine the full range of motion of a joint. Accordingly, the systems and related methods disclosed herein can enable the patient to capture joint ROM data with additional frequency and flexibility, e.g., from the patient's home, without requiring assistance of a trained professional or travel to a specialized location.

Certain exemplary embodiments will now be described to provide an overall understanding of the principles of the structure, function, manufacture, and use of the devices, systems, and methods disclosed herein. One or more examples of these embodiments are illustrated in the accompanying drawings. The devices, systems, and methods specifically described herein and illustrated in the accompanying drawings are non-limiting embodiments. The features illustrated or described in connection with one embodiment may be combined with the features of other embodiments. Such modifications and variations are intended to be included within the scope of the present disclosure.

Additionally, to the extent that linear or circular dimensions are used in the description of the disclosed devices and methods, such dimensions are not intended to limit the types of shapes that can be used in conjunction with such devices and methods. Equivalents to such linear and circular dimensions can be determined for different geometric shapes. Further, in the present disclosure, like-numbered components of the embodiments generally have similar features. Still further, sizes and shapes of the devices, and the components thereof, can depend at least on the anatomy of the subject in which the devices will be used, the size and shape of objects with which the devices will be used, and the methods and procedures in which the devices will be used. To the extent features, sides, components, steps, or the like are described as being “first,” “second,” “third,” etc. such numerical ordering is generally arbitrary, and thus such numbering can be interchangeable.

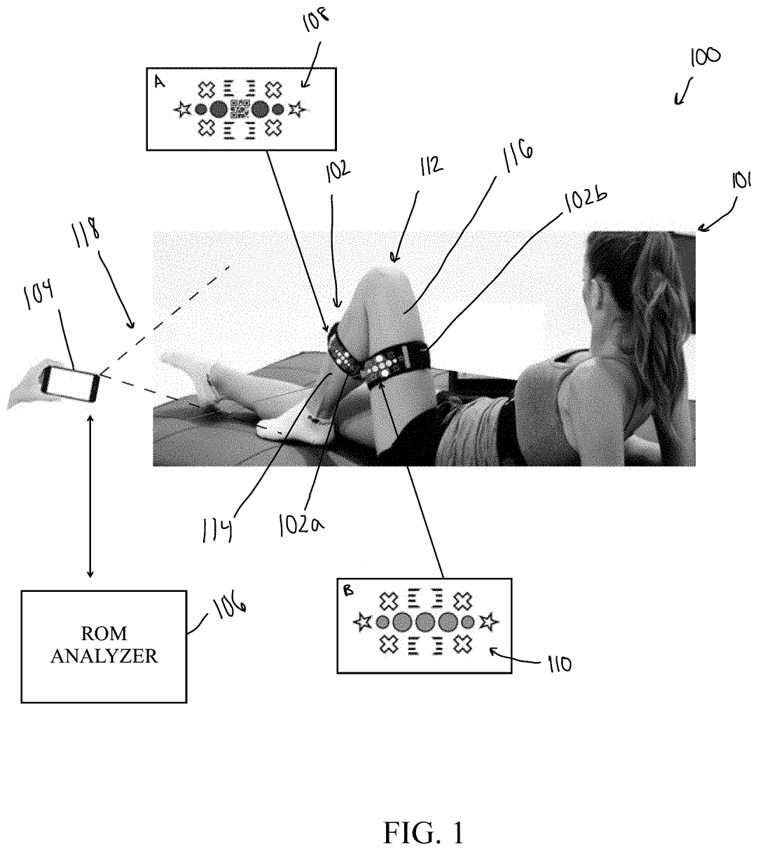

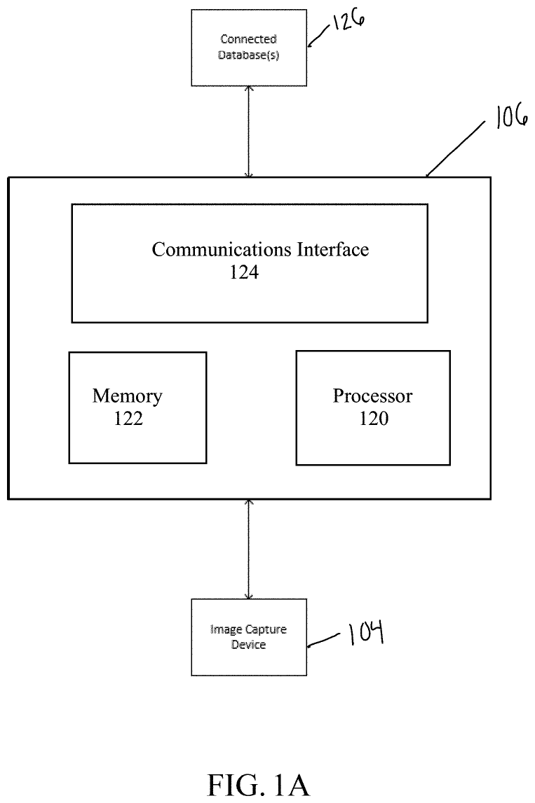

FIG. 1 illustrates one embodiment of a feature-based joint ROM system 100 of the present disclosure that can be used to assess one or more range of motion metrics of a joint 112, e.g., a knee, of a patient 101. The ROM system 100 can include a patient-wearable device 102, an image sensor 104 (also referred to herein as an image capture device), and a range of motion analyzer (ROM analyzer) 106. As noted above, in some embodiments the ROM analyzer can be the local processor on the image capture device 104 while, in other embodiments, the ROM analyzer can be remote from the image capture device and connected thereto via a network. The patient-wearable device 102 can include a first pattern 108 and a second pattern 110, shown in detail in boxes A and B, respectively, that can be coupled to patient anatomy on opposite sides of the joint 112 (e.g., above and below the knee). As discussed in detail below, the first and second patterns 108, 110 can be designed such that the patterns can be recognized and located in an image containing the patterns using, for example, a feature-based image recognition algorithm.

In the illustrated embodiment of FIG. 1, the patient-wearable device 102 can include a first band 102a and a second band 102b. The first pattern 108 can be disposed on the first band 102a and the second pattern 110 can be disposed on the second band 102b. For example, the patterns 108, 110 can be printed on or otherwise securely affixed to the bands 102a, 102b. Each band 102a, 102b can be formed from a flexible material such that the bands can be removably and replaceably secured over a portion of patient anatomy. In some embodiments, the bands can be made from an elastic material that can be slid into place over a portion of patient anatomy. Additionally, or alternatively, the bands 102a, 102b can include a closure mechanism, e.g., a clip, a hook-and-loop fastener, etc., such that the band can be wrapped or adjusted around a portion of patient anatomy and removably secured. The first band 102a can be placed on a first portion 114 of patient anatomy on a first side of the joint 112. The second band 102b can be placed on a second portion 116 of patient anatomy on a second side of the joint 112 that is opposite the first side. Accordingly, the first pattern 108 can be coupled to the first portion 114 of patient anatomy and the second pattern 110 can be coupled to the second portion 116 of patient anatomy such that the first and second patterns are located on opposite sides of the joint 112 from one another.

As used herein, “opposite sides” of a joint can refer to a first location and a second location, with the joint falling between the first and second location, such that there is relative motion of the first and second location upon flexing or extending the joint. For example, where the joint 112 is a knee, as illustrated in FIG. 1, the first portion 114 of anatomy can be located below the knee (e.g., a shin) and the second portion 116 of patient anatomy can be located above the knee (e.g., a thigh), such that the first and second bands 102, 102b and, accordingly, the first and second patterns 108, 110 can be placed on opposite sides of the knee, i.e., below the knee and above the knee, respectively. In some embodiments, the first band 102a can be placed approximately at a mid-point of the patient's shin, the first portion 114 of anatomy, and the second band 102b can be placed approximately at a mid-point of the patient's thigh, the second portion 116 of anatomy.

The image capture device 104 can be used to take at least one image containing the first pattern 108 coupled to the first portion 114 of anatomy, the second pattern 110 coupled to the second portion 116 of anatomy, and the joint 112. The phrase “an image” or “the image,” as used herein in conjunction with embodiments of the present disclosure refers to an image captured with the image capture device 104 containing at least the first pattern 108 coupled to the first portion 114 of patient anatomy on the first side of the joint 112, the second pattern 110 coupled to the second portion 116 of the patient anatomy on the second side of the joint opposite the first side, and the joint. The image capture device 104 can be any device with photo and/or video capture capabilities. In some embodiments, the image capture device 104 can transmit the at least one image to the ROM analyzer 106 for further analysis. By way of non-limiting example, the image capture device 104 can be a mobile device, such as a smartphone or tablet, a laptop, a traditional computer, etc. The at least one image taken by the image capture device 104 can include one or more picture images, one or more video segments, or a combination of the two.

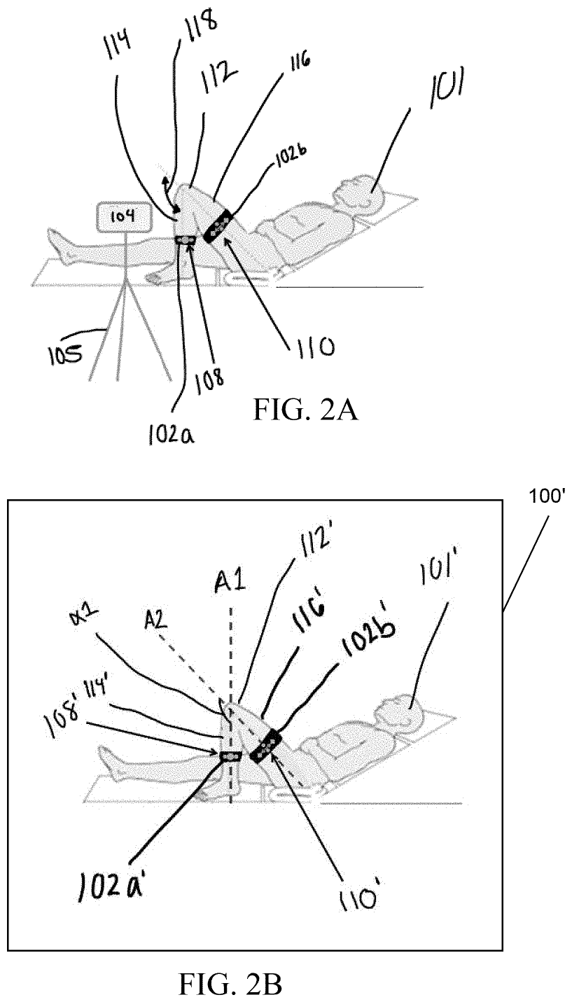

The image capture device 104 can be placed such that the first pattern 108, the second pattern 110, and the joint 112 fall within a viewing range 118 of the image capture device for image capture. In some embodiments, the image capture device 104 can be held by a person at an appropriate distance to place at least the first pattern 108, the second pattern 110, and the joint 112 within the viewing range 118. As noted above, the systems and methods described herein do not require specialized training or assistance to capture an image that can be used to determine at least one range of motion metric. Accordingly, a person holding the image capture device 104 can be, for example, a friend, relative, or caregiver of the patient 101 who does not need to possess any formal training or skills in the medical or imaging fields. Alternatively, the image capture device 104 can be mounted on a tripod 105 (see FIG. 2A), placed on a surface, or otherwise stably positioned to locate at least the first pattern 108, the second pattern 110, and the joint 112 within the viewing range 118. The patterns 108, 110 can be sized such that each pattern can be captured clearly by the image capture device 104 for processing by the ROM analyzer 106. For example, in some embodiments the patterns 108, 110 can be approximately 2 inches in size for clear capture by the image capture device 104 when the image capture device is about 2 feet from the patterns.

FIG. 1A shows a block diagram of the ROM analyzer 106. The ROM analyzer 106 can include at least one processor 120, a memory 122, and a communications interface 124. The processor 120 can include a microcontroller, a microcomputer, a programmable logic controller (PLC), a field-programmable gate array (FPGA), an application specific integrated circuit (ASIC), integrated circuits generally referred to in the art as a computer, and other programmable circuits, and these terms are used interchangeably herein. The processor 120 can be coupled to the memory 122, which can include a random-access memory (RAM), a read-only memory (ROM), a flash memory, a non-transitory computer readable storage medium, and so forth. The memory 122 can store instructions for execution by the processor 120 to implement the systems disclosed herein or to execute the methods disclosed herein. Additionally, or alternatively, the memory 122 can store the information calculated by the processor 120 and/or received by or through the communications interface 124. Although each of these components are referred to in the singular, the various functions described as being carried out by one of the components can be carried out by multiple of these components, e.g., the functions described as being carried out by the processor 120 can be carried out by multiple processors. The communications interface 124 can facilitate communication, i.e., can transmit data between and among, the processor 120, the memory 122, the image capture device 104, and, in some embodiments, one or more connected databases 126. The communications interface 312 can be wireless (e.g., near-field communication (NFC), Wi-Fi, Bluetooth, Bluetooth LE, ZigBee, and the like) or wired (e.g., USB or Ethernet). The communication interface 312 can be selected to provide the desired communication range.

In use, the ROM analyzer 106 can receive at least one image 100′ (see FIG. 2B) from the image capture device 104. The processor 120 can analyze the at least one image 100′ and can determine at least one range of motion metric of the joint 112 based, at least in part, on the image. In some embodiments, the ROM analyzer 106 can transmit information, such as one or more of a calculated range of motion metric, an intermediate calculation based on the at least one image, the at least one image, etc., to the connected database 126. For example, and as discussed in detail below with respect to FIGS. 4-6, the patient wearable device 102 can include one or more information fiducial, which can include a patient-specific marker. In this manner, the information fiducial can be read by the ROM analyzer 106 and can be used to identify a particular patient with which the particular patient-wearable device 102 in the image 100′ is associated. The ROM analyzer 106 can then communicate with the connected database 126, can identify the particular patient based on the information fiducial, and can transmit information, such as the range of motion metric, determined by the processor 120 to the connected database with such information associated with the appropriate particular patient. In some embodiments, one or more components of the ROM analyzer 106 can be integrated within the image capture device 104, such as a smartphone or tablet. Additional details of the ROM analyzer 106 and the joint ROM system 100 will now be described with reference to FIGS. 2A and 2B.

FIG. 2A shows another embodiment of the system 100 in which the image capture device 104 can capture at least one image of the patient-wearable device 102 for a range of motion analysis of the knee joint 112 of the patient 101. FIG. 2B is an image 100′ captured by the image capture device 104 of FIG. 2A. The image 100′ can include a digital representation of at least the first pattern 108′, the second pattern 110′, and the joint 112′. The image 100′ can also include a digital representation of the first portion 114′ of patient anatomy, the second portion 116′, the first band 102a′, and the second band 102b′. The joint ROM system 100 can receive the image 100′ and the processor 120 can determine at least one range of motion metric of the joint 112 based, at least in part, on the image. More particularly, the processor 120 can detect the first and second patterns 108′, 110′ in the image 100′, calculate a longitudinal axis A1, A2 associated with each of the first portion 114′ and the second portion 116′ of patient anatomy, calculate an angle α1 between the axes, and calculate at least one range of motion metric of the joint 112 based on the angle.

The processor 120 can use a feature-based image recognition algorithm, e.g., from the Computer Vision Toolbox™ by MATLAB®, to detect and locate the first and second patterns 108′, 110′ within the frame of the captured image 100′. As an example, the features representing the object can be derived using the Speeded-Up Robust Features (SURF) algorithm. Once the object features are determined, the object can be detected within the captured image 100′ using, for example, the Random Sample Consensus (RANSAC). Each detected pattern 108′, 110′ can be analyzed using, for example, a pattern-recognition algorithm, which can locate centroids of one or more known shapes or elements within the patterns 108, 110. The centroids, or other detected features, can then be used to calculate an orientation of the longitudinal axis A1, A2 for each portion 114′, 116′ of the patient anatomy. The angle α1 between the axes A1, A2 can be calculated, which can represent an angle associated with the joint 112 in a position as captured in the image 100′. A range of motion metric, such as a maximum flexion angle of the joint 112, a maximum extension angle of the joint, and/or a full range of motion, can be determined based on the calculated angle between the longitudinal axes of the first and second portions of patient anatomy.

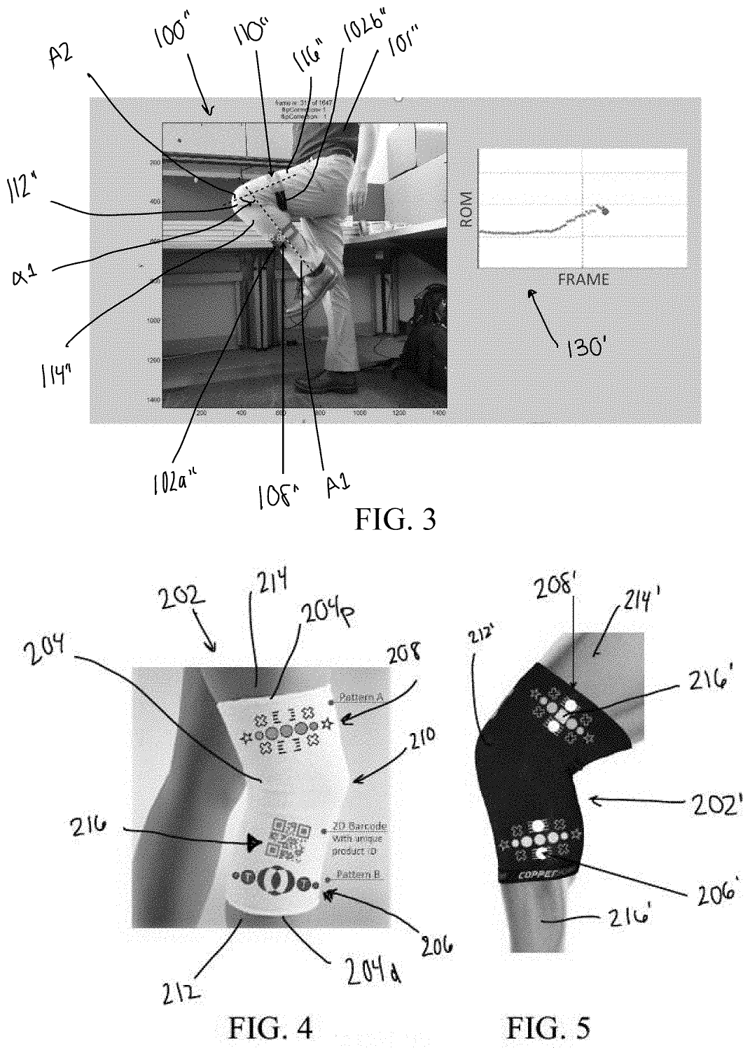

FIGS. 2A and 2B show the patient 101 in a reclined position with the joint 112 in a maximum flexion position. In other embodiments, a patient may be sitting, standing (see FIG. 3), or in any other position as recommended or instructed by a medical professional for use with the system 100. In the illustrated embodiment of FIGS. 2A and 2B, the angle α1 between the first and second portions 114, 114′, 116, 116′ of anatomy can correspond to a maximum flexion angle α1_{Flex} of the joint 112. The first portion 114 of patient anatomy can be moved away from the second portion 116 of patient anatomy, i.e., along the arrow 118 in a direction towards the top of the page of FIG. 2B, to extend the joint 112 and allow for an image to be captured with the joint in an extended position.

The ROM analyzer 106 can use the calculated angle α1 to determine a range of motion metric of the joint 112, such as the maximum flexion angle α1_{Flex}, a maximum extension-angle α1_{Ext}, and/or a full range of motion of the joint. The maximum flexion angle α1_{Flex} can be identified by calculating the angle α1 between the first portion 114 and the second portion 116 from the image 100′ captured when the joint 112 is at maximum flexion, e.g., as shown in FIGS. 1-2B. The maximum extension angle α1_{Ext} can be identified by calculating the angle α1 that extends between the first portion 114 and the second portion 116 of patient anatomy from an image captured when the joint 112 is at maximum extension. The full range-of-motion of the joint 112 can be determined as the difference between the maximum flexion angle α1_{Flex} and the maximum extension angle α1_{Ext}. Accordingly, in some embodiments, the image capture device 104 can capture a first image with the joint 112 at a maximum flexion position and a second image with the joint at a maximum extension position. Additionally, or alternatively, the image capture device 104 can capture a video of the joint 112 moving through an entire range of motion, e.g., moving the joint from a position of greatest extension to a position of greatest flexion or vice-versa. If a video is captured of the entire range of motion of the joint 112, the ROM analyzer 106 can identify a frame of the video corresponding to the position of greatest extension and the position of greatest flexion and can analyze each frame as a discrete image. In some embodiments, the ROM analyzer 106 can receive a plurality of images captured by the image capture device 104 and can analyze each of the images to determine the angle α1 between the first and second portions 114, 116 of anatomy at the time each image was captured, and can subsequently calculate one or more range of motion metrics associated with the joint 112.

As introduced above, FIG. 3 shows another embodiment of an image 100″ captured in accordance with the joint ROM system 100 with the patient 101″ in a standing position and the joint 112″ at maximum flexion. Except as indicated below, the structure, operation, and use of the image 100″ with the joint ROM system 100 is similar or identical to that of the image 100′, with like-numbered components generally having similar features. Accordingly, description of the structure, operation, and use of such features is omitted herein for the sake of brevity. The ROM processor 106 can receive the image 100″ and can detect the first and second patterns 108″, 110″ within the image. Central longitudinal axes A1, A2 can be located based on the detected patterns 108″, 110″, and the angle α1 extending between the axes can be calculated. The ROM analyzer 106 can calculate one or more range of motion metrics of the joint 112″ based on the calculated angle α1, as described above. In some embodiments, the image capture device 104 can capture a video segment that can include motion of the joint 112″ from a point of maximum flexion to a point of maximum extension. The ROM analyzer 106 can perform the calculations described herein and can calculate an angle α1 extending between the longitudinal axes A1, A2 of the first and second portions of patient anatomy, as captured by the image capture device 104, for each frame (i.e., image) of the video. In some such embodiments, the ROM analyzer 106 can output a graphical representation 130′ of a range of motion of the joint 112″.

FIG. 4 shows another embodiment of a patient-wearable device 202 in accordance with the present disclosure, which can be used in the joint ROM system 100 in a similar manner as the patient-wearable device 102 illustrated in FIG. 1. The patient-wearable device 202 can be formed as a single or unitary sleeve 204 with a first pattern 206 disposed on a distal portion 204d of the sleeve and a second pattern 208 disposed on a proximal portion 204p of the sleeve. The sleeve 204 can be made from an elastic or other flexible material, such that the sleeve can be placed over a joint 210, e.g., a knee, with the distal portion 204d of the sleeve placed over a first portion 212 of patient anatomy (e.g., a shin) on a first side of the joint and the proximal portion 204p of the sleeve placed over a second portion 214 of patient anatomy (e.g., a thigh) on a second side of the joint opposite the first side. Accordingly, the first pattern 206 can be coupled to the first portion 212 of patient anatomy on the first side of the joint 210 and the second pattern 208 can be coupled to the second portion 214 of patient anatomy on the second side of the joint opposite the first. The image capture device 104 and the ROM analyzer 106 can be used as described above with respect to the system 100 in FIG. 1 to determine at least one range of motion metric of the joint 212.

The patient-wearable device 202 can include an information fiducial 216 disposed on the sleeve 204. By way of non-limiting example, the information fiducial 216 can be a two-dimensional barcode, e.g., a QR (quick response) code, that can include a unique product information number to identify the particular patient-wearable device 202. In some embodiments, such as the embodiment illustrated in FIG. 4, the information fiducial 216 can be distinct from the first pattern 206 and the second pattern 208. In other embodiments, one or more information fiducials 216 can be incorporated into the first and/or second patterns 206, 208. A location of the information fiducial 216, e.g., on the distal portion 204d of the sleeve, can assist the patient in identifying an intended orientation of the sleeve. In some embodiments, the information fiducial 216 can be a patient-specific marker such that a particular wearable device 102, 202 can be associated with a particular patient. For example, the information fiducial 216 can include the unique product information for a particular wearable device 102, 202. The information fiducial 216 can registered or otherwise linked to a particular patient upon issuing the wearable device 102, 202 to a patient, e.g., by scanning the information fiducial and associating the scanned information with a patient profile in a patient database. In use, the ROM analyzer 106 can link a range of motion metric to an appropriate patient profile by reading the information fiducial 216 within a captured image. The processor 120 can associate the at least one range of motion metric calculated by the ROM analyzer 106 and transmit the information to the connected database 126, which can, for example, store the range of motion metric with the appropriate patient profile and/or the particular patient-wearable device 102, 202.

FIG. 5 illustrates another embodiment of a patient-wearable device 202′ of the present disclosure. The patient-wearable device 202′ is a variation of the patient-wearable device 202 of FIG. 4. Accordingly, except as indicated below, the structure and use of this embodiment is similar or identical to that of the patient-wearable device 202′, with like-numbered components generally having similar features. The wearable device 202′ of FIG. 5 can be formed as a single or unitary sleeve 204′ with a first pattern 206′ disposed on a distal portion 204d′ of the sleeve and a second pattern 208′ disposed on a proximal portion 204p′ of the sleeve. The sleeve 204′ can be placed over a joint 210′, e.g., a knee, such that the first pattern 206′ can be coupled to a first portion 212′ of patient anatomy (e.g., a shin) on a first side of the joint 210′ and the second pattern 208′ can be coupled to a second portion 214′ of patient anatomy (e.g., a thigh) on a second side of the joint opposite the first. The image capture device 104 and the ROM analyzer 106 can be used as described above with respect to the system 100 in FIG. 1 to determine at least one range of motion metric of the joint 212′. An information fiducial 216′ can be incorporated within the second pattern 208′ on the proximal portion 204p′ of the sleeve 204′. For example, the information fiducial 216′ can be a two-dimensional barcode placed centrally within the second pattern 208′.

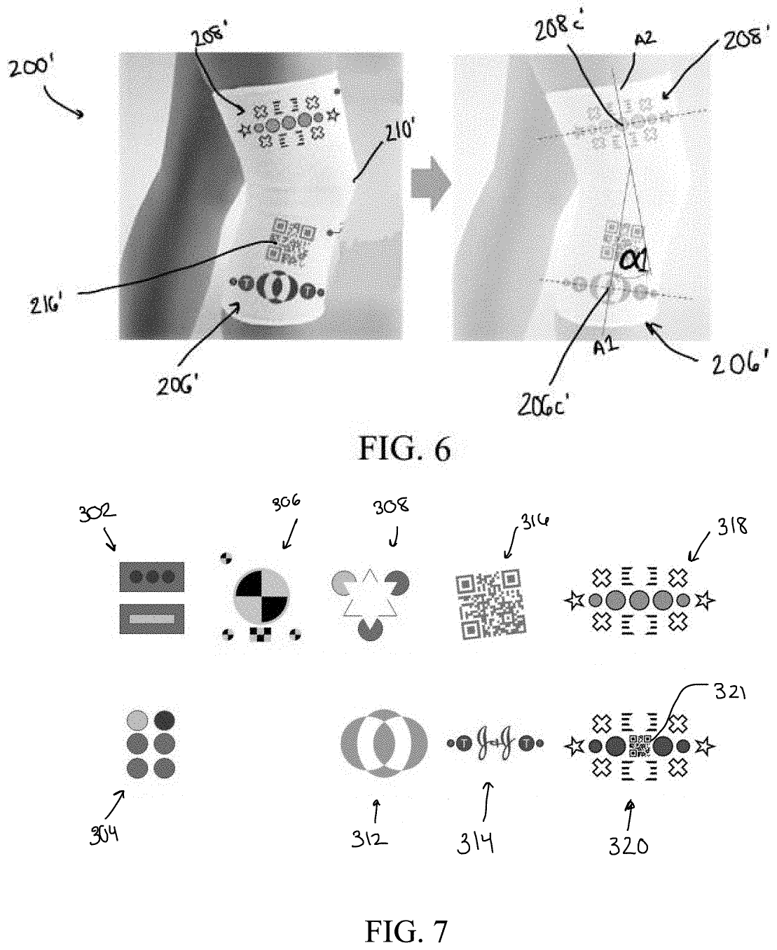

FIG. 6 illustrates calculations that can be performed by the ROM analyzer 106 from an image 200′ captured with the image capture device 104 of the first pattern 206, second pattern 208, and joint 210 in the embodiment illustrated in FIG. 4 in conjunction with calculating one or more range of motion metric of the joint 210. More particularly, the ROM analyzer 106 can detect a centroid 206c′, 208c′ of the first and second patterns 206′, 208′ captured in the image 200′. Based on the detected centroids 206c′, 208c′, the ROM analyzer 106 can locate the patterns 206′, 208′ and calculate an angle and orientation of a central longitudinal axis A1, A2 of the first and second portions of anatomy 212, 214 to which the patterns 206′, 208′ can be coupled. As described above, an angle α1 extending between the longitudinal axes A1, A2 can be calculated which can represent an angle of the joint 210′ in the position as captured in the image 200′, which can then be used to determine one or more range of motion metric of the joint.

As can be seen from the various embodiments of patient-wearable devices 102, 202, 202′ illustrated herein, first and second patterns of the present disclosure can have varying configurations, both with respect to one another (i.e., a first pattern and a second pattern associated with a particular patient-wearable device can be unique from one another) and/or across patient-wearable devices (i.e., a first pattern of a first wearable device can have a different configuration than a first pattern of a second wearable device). The systems and methods disclosed herein can use a feature-based approach to detect and locate the first and second patterns within an image. Features that can be recognized in an image to locate patterns within an image can include, for example, points, edges, objects, shapes defined in terms of curves or boundaries between different image regions, etc. Accordingly, the first and second patterns 108, 110 can be designed based, at least in part, on striking a balance between a number of recognizable features, space constraints on the patent-wearable device, and/or cost of producing the pattern(s). Patterns with certain elements or features, such as sharp edges and/or high contrast, can improve the effectiveness and ease with which the pattern can be identified with the feature-based algorithm.

In the embodiment illustrated in FIG. 1, the patterns 108, 110 each contain complex, highly recognizable features, such as sharp edges and high contrast. Alternative configurations of one or more of the patterns 108, 110, however, fall within the scope of this disclosure. FIG. 7 illustrates non-limiting embodiments of patterns that can be used in association with patient-wearable devices of the present disclosure. In some embodiments, a pattern can include an arrangement of one or more basic shapes, placed in a manner such that a feature-based recognition algorithm can identify the pattern in a captured image. For example, a pattern 302 can include two or more elements of different geometric shapes, e.g., rectangles and circles of varying size and color. Alternatively, a pattern 304 can include a number of geometric elements having the same basic shape and size but of various colors. For example, in the illustrated embodiment of one such pattern 304, the pattern can include two columns of three-circles each, with each of the circles in a column having a different color from any adjacent circle. While the geometric elements in the patterns 302, 304 each have a solid-fill, in other embodiments one or more geometric element may be of varying color. For example, a pattern 306 can include one or more geometric element with different colored sections within the element. In some embodiments, the pattern 306 can include a geometric elements of varying shapes and/or size that can have alternating yellow and black sections within the element, reminiscent of a crash test marker. Multi-color patterns with more complex arrangement of geometric shapes can also be used, as shown, for example, in pattern 308 that can include an overlapping arrangement of triangles and circles, with each circle having a different color.

In some embodiments, a pattern can include one or more abstract elements, pictures, or symbols. For example, a pattern 312 can include an abstract design that can have a desired aesthetic and/or style, while incorporating certain features recognizable by the ROM analyzer 106. A logo or other branding mark can be used as a stand-alone pattern 312, e.g., a DePuy Synthes Companies logo, or can be incorporated among other elements to form a pattern 314, e.g., inclusion of a Johnson & Johnson logo with additional elements to form the pattern. As discussed above, an identification fiducial 316 can be used as a pattern, such as a two-dimensional (2D) barcode. The identification fiducial 316 can contain information particular to the specific wearable-device and/or patient to whom the wearable-device is given. For example, the identification fiducial can include unique identification data specific to a particular wearable-device. In this manner, the identification fiducial can be scanned, for example by a healthcare professional at the time a wearable-device is given to a patient, to link the device to a profile or identification number of the patient in a digital health platform. Accordingly, in some embodiments, the ROM analyzer 106 can associate one or more ROM metrics calculated from an image of a particular wearable-device having the identification fiducial 316 with the appropriate patient. Finally, FIG. 5 includes two embodiments of complex patterns 318, 320 that can include a plurality of features that can be highly recognizable to a feature-based recognition algorithm, such as strong edges and high contrast. In some embodiments, the complex pattern 320 can incorporate an identification fiducial 321 within the pattern.

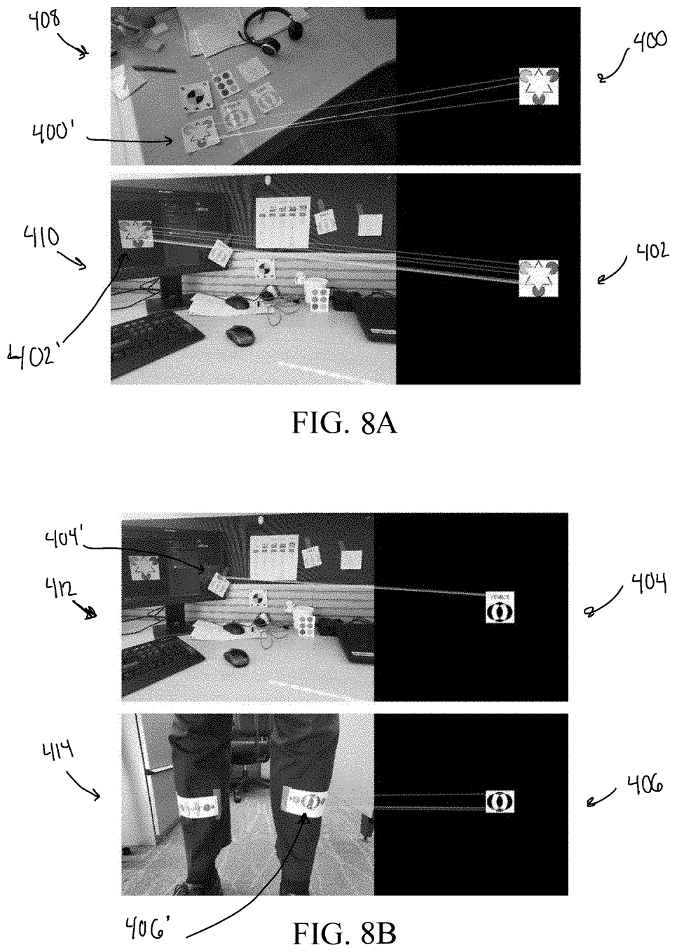

The feature-based approach to image recognition can be more robust as compared to template matching or image cross-correlation. For example, the feature-based approach can have improved occlusion tolerance, as compared to other image recognition constructs, and can be invariant to scale and/or rotation of an image. FIGS. 8A and 8B show examples of detected patterns 400, 402, 404, 406 recognized using a feature-based recognition algorithm from an image 408, 410, 412, 414 that includes the patterns 400′, 402′, 404′, 406′ within a frame of the image. The detected patterns 400, 402 in FIG. 8A can include a multi-colored assortment of geometric shapes in a somewhat complex arrangement, e.g., similar to the pattern 308 of FIG. 7. As illustrated in FIG. 8A, the feature-based recognition algorithm can successfully detect patterns 400, 402 with fidelity to the pattern 400′, 402′ captured in an image 408, 410, despite differences in scale of the patterns 400′, 402′ relative to other elements within the frame of the captured images 408, 410, respectively. FIG. 8B illustrates the capability of the ROM analyzer 106 using the feature-based image recognition approach to successfully detect patterns 404, 406 from images 412, 414 in which the patterns captured in the image 404′, 406′ are in a rotated position. Moreover, FIGS. 8A and 8B illustrate that the ROM analyzer 106, with feature-based image recognition, can successfully detect patterns 400, 402, 404, 406, notwithstanding deformation to the pattern, e.g., due to deformation of a fabric to which the pattern is attached, poor or inconsistent lighting conditions, rotation of the pattern, etc.

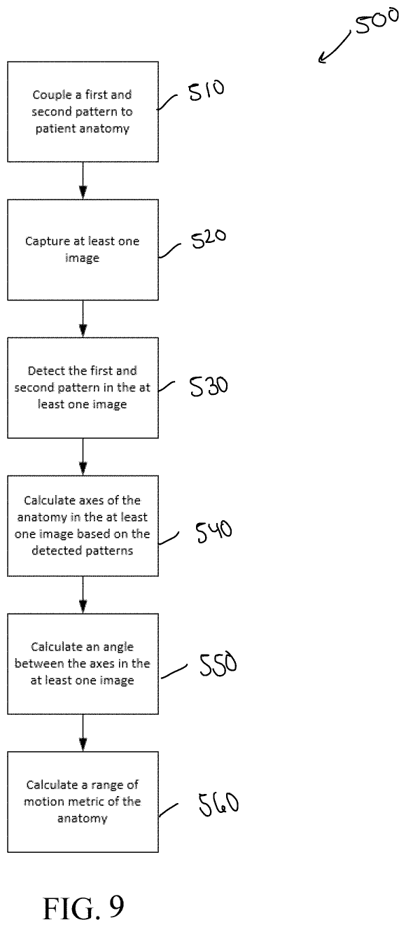

FIG. 9 illustrates one embodiment of a method 500 for determining a range of motion metric for a joint in accordance with the present disclosure, which can be performed with any of the systems and devices described herein. The method can include coupling a first pattern to a first portion of patient anatomy on a first side of a joint and coupling a second pattern to a second portion of anatomy on a second side of the joint opposite the first side 510 and capturing at least one image including the first pattern, the second pattern, and the joint within the frame of the image 520. As discussed above, the steps of coupling the first and second patterns to patient anatomy (510) and capturing the at least one image (520) can be performed in a patient's home, or other desired location, by the patient without the assistance of a trained professional. The method can further include detecting the first and second pattern in the at least one image 530, calculating axes of the first and second portions of anatomy in the at least one image based on the detected patters 540, calculating an angle between the axes in the at least one image 550, and calculating a range of motion metric 560 associated with the joint in the at least one image. In some embodiments, the step of capturing at least one image 570 and calculating a range of motion metric based, at least in part, on the at least one image (i.e., steps 530-560) can be repeated a plurality of times. For example, as described above, a first image can be captured in which the joint is in a maximum flexion position and a second image can be captured in which the joint is in a maximum extension position. A range of motion metric can be calculated from each of these images or from a combination of images, e.g., through steps 530-560 of the method 500.

Although specific embodiments are described above, changes may be made within the spirit and scope of the concepts described. For example, the above embodiments describe a knee joint range-of-motion application. While this is one contemplated use, the methods and devices of the present disclosure can be equally adapted for use in other areas of a patient's body, e.g., an elbow joint, wrist joint, ankle joint, etc. As such, the devices described herein can be formed in a variety of sizes and materials appropriate for use in various areas of a patient's body. Accordingly, it is intended that this disclosure not be limited to the described embodiments, but that it have the full scope defined by the language of the claims. All publications and references cited herein are expressly incorporated herein by reference in their entirety.

1. A system for measuring joint range of motion, comprising:
a first pattern configured to be coupled to a first portion of anatomy of a patient on a first side of a joint;
a second pattern configured to be coupled to a second portion of anatomy of the patient on a second side of the joint opposite the first side;
an image sensor configured to capture at least one image containing the joint, the first pattern, and the second pattern; and
a processor configured to, for one or more of the at least one images, recognize the first pattern and the second pattern, calculate axes of the first and second portion of anatomy to which the first and second patterns are coupled, and calculate an angle between the axes;
wherein the processor is further configured to calculate at least one range of motion metric based on the calculated angle between the axes in the at least one image.

2. The system of claim 1, wherein the range of motion metric is a full range of motion and the at least one image includes a first image in which the joint is at maximum extension and a second image in which the joint is at maximum flexion, and
wherein the processor is configured to calculate the full range of motion as a difference between the angle between the axes when the joint is at maximum extension and the angle between the axes when the joint is at maximum flexion.

3. The system of claim 1, wherein the range of motion metric is a maximum extension angle and the at least one image includes an image in which the joint is at maximum extension, and
wherein the processor is configured to calculate the maximum extension angle as the angle between the axes when the joint is at maximum extension.

4. The system of claim 1, wherein the range of motion metric is a maximum flexion angle and the at least one image includes an image in which the joint is at maximum flexion, and
wherein the processor is configured to calculate the maximum flexion angle based on the angle between the axes when the joint is at maximum flexion.

5. The system of claim 1, wherein the first portion of anatomy is a shin, the second portion of anatomy is a thigh, and the joint is a knee.

6. The system of claim 1, wherein the image sensor and the processor are contained within a smartphone or tablet.

7. The system of 1, wherein the first pattern is coupled to a first elastic band configured to be placed over the first portion of anatomy and the second pattern is coupled to a second elastic band configured to be placed over the second portion of anatomy.

8. The system of claim 1, wherein the first pattern is disposed on a proximal portion of an elastic sleeve and the second pattern is disposed on a distal portion of an elastic sleeve, the elastic sleeve being configured to be placed over the joint, the first portion of anatomy, and the second portion of anatomy.

9. The system of claim 1, wherein at least one of the first pattern and the second pattern includes at least one patient-specific marker; and
wherein the processor is further configured to identify the patient-specific marker with a particular patient and communicate with an associated database based on the identified particular patient

10. A method for measuring joint range of motion, comprising:
coupling a first pattern to a first portion of anatomy of a patient on a first side of a joint;
coupling a second pattern to a second portion of anatomy of the patient on a second side of the joint opposite the first side;
capturing at least one image containing the joint, the first pattern, and the second pattern;
detecting the first pattern and the second pattern for each of the at least one image;
calculating axes of the first and second portions of anatomy based on the detected patterns in the at least one image;
calculating an angle between the axes in the at least one image; and
calculating a range of motion metric based on the calculated angle between the axes in the at least one image.

11. The method of claim 10, wherein capturing at least one image includes capturing a first image in which the joint is at maximum extension and a second image in which the joint is at maximum flexion.

12. The method of claim 11, wherein the range of motion metric is a full range of motion and calculating the range of motion metric further includes:
calculating an angle between the axes of the first image;
calculating an angle between the axes of the second image; and
calculating the range of motion as a difference between the angle between the axes when the joint is at maximum extension and the angle between the axes when the joint is at maximum flexion.

13. The method of claim 10, wherein the at least one image includes an image in which the joint is at maximum extension and the range of motion metric is a maximum extension, and calculating the range of motion metric further comprises calculating the maximum extension as the angle between the axes when the joint is at maximum extension.

14. The method of claim 10, wherein the at least one image includes an image in which the joint is at maximum flexion and the range of motion metric is a maximum flexion, and calculating the range of motion metric further comprises calculating the maximum flexion as the angle between the axes when the joint is at maximum flexion.

15. The method of claim 10, wherein the first portion of anatomy is a shin, the second portion of anatomy is a thigh, and the joint is a knee.

16. The method of claim 10, wherein a smartphone or tablet is used to capture the at least one image.

17. The method of claim 16, wherein the smartphone or tablet is used to detect the first and second patterns in the at least one image, calculate the axes of the first and second portions of anatomy, calculate the angle between the axes, and calculate the range of motion metric.

18. The method of claim 16, wherein capturing the at least one image further includes capturing a video segment using the smartphone or tablet.

19. The method of claim 10, wherein detecting the first pattern and the second pattern for the at least one image is performed using a feature-based image recognition algorithm.

20. A system for capturing a joint range of motion, comprising:
one or more processors of a range of motion platform on a network, the one or more processors configured to:
receive at least one image taken with a smartphone or tablet, the image including a joint, a first pattern coupled to a first portion of anatomy on a first side of the joint, and a second pattern coupled to a second portion of anatomy on the second side of the of the joint;
detect the first pattern and the second pattern for the at least one image;
calculate axes of the first and second portions of anatomy based on the detected patterns in the at least one image;
calculate an angle between the axes in the at least one image; and
calculate a range of motion metric based on the calculated angle between the axes in the at least one image.

21. The system of claim 20, wherein the range of motion metric is a full range of motion and the at least one image includes a first image in which the joint is at maximum extension and a second image in which joint is at maximum flexion, and
wherein the one or more processor configured to calculate the range of motion as a difference between the angle between the axes when the joint is at maximum extension and the angle between the axes when the joint is at maximum flexion.

22. The system of claim 20, wherein at least one of the first pattern and the second pattern includes at least one patient-specific marker; and
wherein the one or more processors are further configured to identify the patient-specific marker with a particular patient and communicate with an associated database based on the identified particular patient.
